(19) United States
(12) Patent Application Publication
(10) Pub. No.: US 2012/0282681 A1
(43) Pub. Date: Nov. 8, 2012

(54) Rapid Identification of Organisms in Bodily Fluids

(76) Inventors: Scott M. Teixeira, Cumming, GA (US); Adrìenne A. Hershey, Cumming, GA (US); Brian J. Cuevas, Cumming, GA (US); Joseph A. Cesa, Cumming, GA (US); Amy G. Williams, Cumming, GA (US); Thomas D. Haubert, Columbus, OH (US); Jeffrey R. Held, Columbus, OH (US); James B. Gleeson, Columbus, OH (US); Stephen C. Schmitt, Dublin, OH (US)

(21) Appl. No.: 13/459,576

(22) Filed: Apr. 30, 2012

Related U.S. Application Data
(60) Provisional application No. 61/482,773, filed on May 5, 2011.
(60) Provisional application No. 61/596,838, filed on Feb. 9, 2012.

(51) Int. Cl. C12M 1/34; B01L 3/00

(52) U.S. Cl. 435/287.2; 422/557; 435/288.7

(57) ABSTRACT

There is provided a device that retains a collected sample for on-demand testing of a small portion of the collected sample while the rest of the sample remains for optional additional analysis. The on-demand test provides relatively immediate information about aspects of the sample, e.g. presence of microbes, chemistry, nutritional condition, presence of contaminants.

This application claims the benefit of U.S. provisional patent applications 61/482,773 and 61/596,838.

The present disclosure relates generally to the field of medicine and more particularly relates to a device that can identify bacterial type above a certain threshold concentration.

When a patient is admitted to a hospital, or a specific unit of the hospital, e.g.; the ICU (intensive care unit), they are often tested for the presence of infection-causing microorganisms in their system through blood, urine, skin, and sputum. Depending on hospital protocol this screening test is completed upon admission to the various areas of the hospital or upon clinical signs of infection including fever, increased white blood cell count, discolored sputum, purulent sputum, decreased oxygenation, hazy chest X-ray, etc.

Currently, the sputum samples are obtained via bronchoscopy, non-bronchoscopic broncheoaviolar lavage (BAL), closed suction catheter, open suction catheter, or another collection apparatus 16 as indicated in FIG. 1, or from an expectorated sample. The sample is then retained in a container 10 that is often connected to the apparatus 16 through flexible tubing connections 12, 14 or other means. Current containers are prone to leakage or spillage, causing concern to the medical personnel involved since the exact microorganisms present are unknown. The disconnection of tubing from current containers is also a source for leakage.

The container holding the sample is transported to the clinical microbiology laboratory for microbial testing and analysis. The container is commonly transported in a pneumatic tube system from the ICU to the lab. A problem that sometimes arises is that the sample can spill or leak in the pneumatic tubing as it is being transported. This can contaminate the pneumatic system, putting the integrity of other samples transported at risk and requiring a re-sampling of the patient, with its concomitant risks.

While the clinician is waiting for the microbial data to return and the patient is showing clinical signs of infection, common practice is to give the patient 3-5 broad spectrum antibiotics to cover all possible organisms that could be causing the infection. These antibiotics have toxic side effects for the patient. For example, some antibiotics can cause harm to the function of the kidneys. Overuse of unnecessary antibiotics can cause “super bugs” and antibiotic resistance, which is a well published problem in health care. The use of these potentially unnecessary antibiotics also incurs a large cost to the hospital. The clinician may also isolate a patient that is suspected of having a resistant or highly contagious organism (e.g.; MRSA or TB). There is, of course, an associated cost to so isolate a patient suspected of carrying a concerning organism.

The first round of microbial data that a physician receives is called a gram stain. A gram stain identifies if a bacterial organism is in either the gram negative or gram positive class and the morphology of the bacteria (i.e. cocci, rod, etc. . . . ). This allows the clinician to remove antibiotic(s) that affect the class of organisms with which the patient is not infected. A gram stain test takes approximately 1 hour to perform, but with transportation time of the sample and the typical lab testing back-log, most ICU clinicians receive the gram stain results in 12-24 hours. During this time a patient is placed on the 3-5 broad spectrum antibiotics mentioned above until the clinician reviews the gram stain results and removes 1-3 unnecessary broad spectrum antibiotics.

Many studies have tested the specificity and sensitivity of the standard gram stain and the general consensus is that the gram stain in about 80% sensitive and 80% specific. The gram stain is a subjective test because the lab technician is viewing the sample under a microscope to identify the color and location of a staining dye in bacteria cells and tests results could be gram variable, meaning the technician could not identify the bacterial gram class. There are also several steps to complete a gram stain that include chemical washings and dyes that are user dependent. If these steps are not followed well, the test could be less accurate. The gram stain procedure generally includes the followings steps: 1) place a slide with a bacterial smear on a staining rack, 2) stain the slide with crystal violet for 1-2 minutes, 3) pour off the stain, 4) flood slide with Gram's iodine for 1-2 min., 5) pour off the iodine, 6) decolorize by washing the slide briefly with acetone (2-3 seconds), 7) wash slide thoroughly with water to remove the acetone—do not delay with this step, 8) flood slide with safranin counter stain for 2 min., 9) wash with water, 10) blot excess water and dry by hand over (Bunsen) flame.

The second round of microbial data that a physician receives is called a microbial specificity. These results are usually obtained in 24-48 hours and require culturing of the organisms on an agar plate. Microbial specificity identifies the exact organism(s) that are causing the infection and the concentration of that organism(s) in a quantitative or semi-quantitative fashion. These results allow the clinician to change the broad spectrum antibiotics to antibiotics targeted for the specific organism that is causing the infection. The clinician may also wait to change antibiotics if the patient is improving or until further results are obtained.

The third round of microbial data that a physician receives is call antibiotic sensitivities. These results are obtained in 48-72 hours and require testing the cultured sample against known antibiotics to determine the resistance pattern of the organism. Once it is known what antibiotics the organism is sensitive to or will kill the organism(s), the clinician can change to one or at least fewer targeted antibiotic to treat the infection.

Thus, there remains a need in the art for a device that retains a collected sample yet analyzes a portion of the sample per an on-demand test system that is easy enough to be performed at the bedside to give the physician timely information about the condition of a patient. This will reduce the time it takes for the physician to receive test results and make better antibiotic prescription choices that should lead to decreased antibiotic resistance, decreased toxicity to the patient, improved patient outcome, and saved time in beginning proper treatment.

### SUMMARY

In response to the difficulties and problems discussed herein, the present disclosure provides a sample isolation and on-demand testing device (equivalently termed “the device”). The device includes: a receptacle, termed “sample cup”, or “cup” to retain a sample; other subsequently described components for on-demand testing of a small portion of the collected sample, equivalently termed “sample portion” or “portion of the sample”; retention of the remaining sample in the cup for optional additional analysis. The on-demand test provides relatively immediate information about aspects of the sample, e.g. presence of microbes, chemistry, nutritional condition, presence of contaminants. One exemplary on-demand test would determine the presence of gram negative bacteria, gram positive bacteria, both gram negative and gram positive bacteria, or no bacteria detected. Such a test is envisioned as clinically significant when the detection level would be above a specific bacterial concentration threshold to indicate infection (i.e. 10̂3 cfu/ml) versus the presence of a bacteria that is not a part of the infection.

According to this disclosure, a non-bronchoscopic or bronchoscopic collection apparatus may be used to obtain a sample, e.g. sputum, from the patient. When the sample is sputum, desirably the collection apparatus obtains the sample below the corina and ideally in the third generation lung lobe. Such a sample is deposited in the cup of the inventive device. Desirably the collection apparatus is integrated with the device. The test and all the components and additives needed to complete the test are desirably completely integrated into the device so no secondary processing is needed. If other steps, such as mixing and pipetting additives were needed, this test would not be practical to perform at the bedside.

This device could also be used as a screening tool to test a patient upon admittance to the hospital or admission to a specific unit of the hospital to determine if the patient is colonized with a clinically significant concentration of bacteria. This information would allow the clinician to isolate or treat a patient before clinical signs of infection are obvious. This early information could also help a hospital determine if a patient obtained a hospital acquired infection (HAI) or already had an infection prior to admission, called a community acquired infection (CAI) for public reporting and billing purposes.

The general manner in which the device functions is: a sample obtained via a sampling catheter or bronchoscope or other means, desirably attached to the device, is deposited in the cup; a portion of the sample is admitted into a chamber that defines a pre-determined sample portion volume, the sample portion is isolated from the balance of the sample so that the rest of the sample remains as collected for optional additional testing; the isolated sample portion is conducted towards an assay assembly and, if required, has an additive (e.g. to lyse) or additives added; the sample portion and any additives are directed to the assay assembly that performs a test; upon completion of the test a detector communicates the results of the test to an externally visible display.

The additive and sample portion are desirably well mixed and this could be activated and timed by a mechanical button or slide or via an additional button push, passive and or active valve, mechanical motor, or other means. Such mixing may also take place by simultaneously directing the sample portion and additive through a common port to the assay assembly. Once the optional additive and the sample portion are mixed, they are conducted to directly contact the assay assembly. The device allows for adequate exposure of the sample portion and any necessary mixed additive to the assay assembly for a pre-determined period of time to ensure migration of some of the sample portion with mixed additive through the assay assembly. An example of such a time period is desirably about 15 minutes or even less, though preferably less than 30 minutes. At the end of that time period, a detector may sense a change in a colorimetric, florescent, magnetic or other expression of a test result in the assay assembly. The detector outputs the result to a display for external visualization. An example of a detector that is suitable for use in the device is an optical detector that scans for changes in reflected light at specific intensity ranges. When such a detector is used, the scanned intensity of the test expression is an important factor in determining the output to the display; when a scanned intensity is outside a specific range, such a detector is programmed to prevent the detector from outputting erroneous results, e.g. slight cross reactivity of the test. The display is important so the user (e.g. nurse or respiratory therapist) has no subjectivity in interpreting the test results.

By providing a reliable sample and rapid bed-side results, this device allows the clinician to potentially prescribe fewer initial antibiotics to the patient, thus reducing toxicity for the patient, decreasing antibiotic resistance, and saving the hospital costs on unnecessary antibiotics.

### BRIEF DESCRIPTION OF THE DRAWINGS

FIG. 1 is a drawing of a prior art sample collection container.

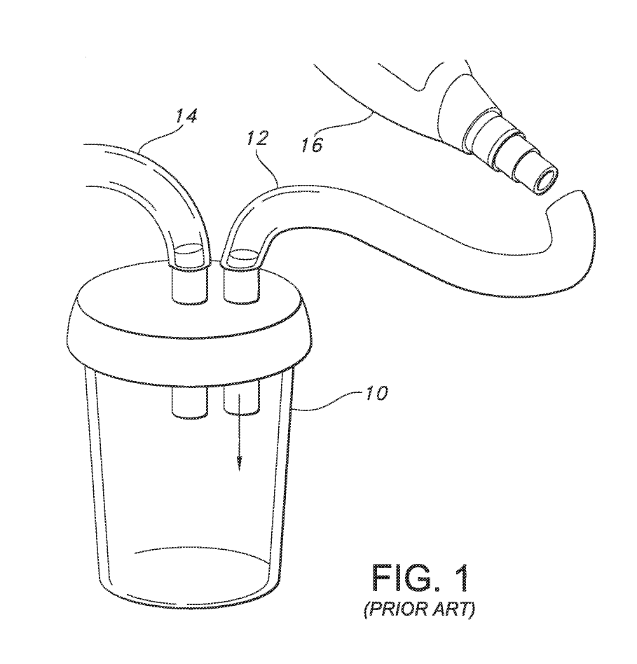

FIG. 2 is a representation of a lateral flow assay strip, illustrating the various layers and components that are used to construct the strip.

FIG. 3 is a drawing of an embodiment of a sample cup and an assay assembly according to the disclosure where the assembly performs one test.

FIG. 4 is an exploded view of the embodiment of FIG. 3.

FIGS. 5A, 5B and 5C are drawings of different ways in which the results of the disclosed test may be displayed.

FIG. 6 is a drawing of an embodiment of a sample cup and assay assembly according to the disclosure where the assembly performs two tests.

FIG. 7 is an exploded view of the embodiment of FIG. 6.

FIGS. 8A and 8B show a side view (8A) and a top view (8B) of another embodiment according to the disclosure that shows a different orientation of the components of the device from those shown in FIGS. 3 and 6.

FIGS. 9A and 9B are alternate views of FIG. 8 showing a cutaway side view (9A) and a top view (9B) of the device prior to use.

FIG. 10 is a close up view inside a portion of the cup showing an opening to the chamber from the cup and ribs that may filter out large particles.

FIGS. 11A, 11B and 11C are alternate views of FIG. 8 showing a cutaway side view (11A), a top view (11B) of the device at the position where it is almost completely activated and an intermediate position (11C).

FIGS. 12A, 12B and 12 C show partial top (12A) and side cutaway views of an embodiment of the device. In FIG. 12C the device is not activated and the chamber and cup are in fluid communication. In FIG. 12B the device is activated and fluid has been moved by a piston to an assay assembly via an outlet port.

FIGS. 13A and 13B are partial top (13A) and side (13B) cutaway views of an embodiment with a piston and a check valve. FIG. 13C shows the device activated but prior to the puncturing of the additive foil packet. FIG. 13D shows the device when it is almost completely activated and the foil packet has been punctured.

FIGS. 14A, 14B and 14C are partial top (14A, 14C) and side (14B) cutaway views of an embodiment without the containment structure for the additive present. FIG. 14C shows the device prior to activation and 14A and 14B show the device after activation.

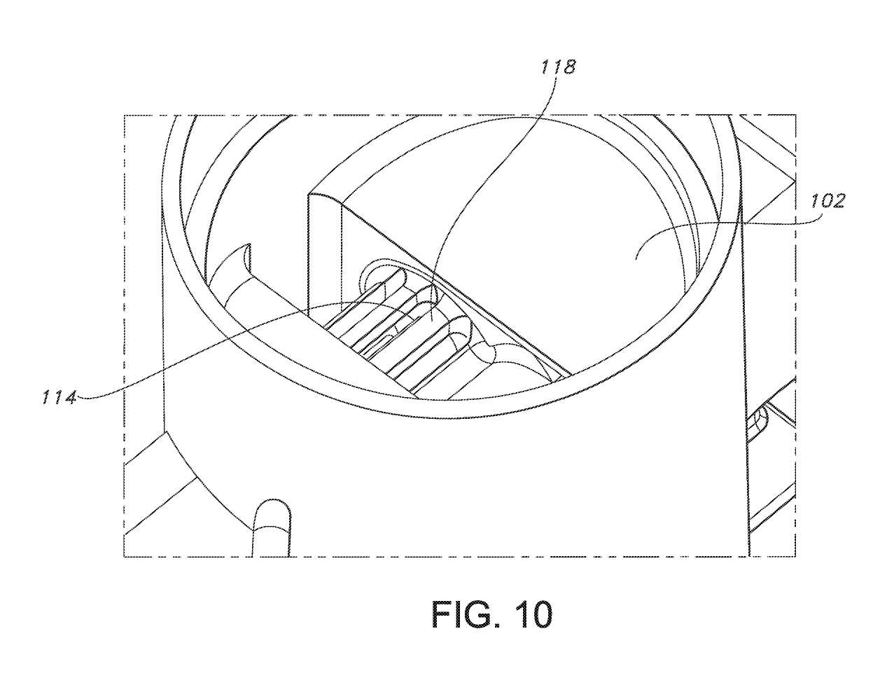

It should be noted that FIGS. 9 and 11-14 show depictions of the device from the top without the assay assembly shown.

### DETAILED DESCRIPTION

The mechanical components of this device are required to contain a sample, separate a portion of the sample, add an optional additive to the sample portion, optionally wait a specified delay time, and then conduct the sample portion and additive to the assay assembly. Inherent in all the steps will be sealing to provide segregation of fluids and prevention of exposed biohazards. Additionally, the product desirably has a 1 to 2 year shelf life (dependent on the assay assembly), which requires solutions for seal performance and prevention of premature fluid migration. Finally, the clinician prefers to perform a single action to activate the device, and not more than two actions.

It is important to segregate the sample portion to be tested from the rest of the sample in the cup in order to preserve the rest of the sample for further testing if necessary. Movement of the sample portion, ˜1 to 5 ml, into a sample chamber while the cup and chamber are in fluid communication can be accomplished by gravity, suction, or other means. Gravity can be a reasonable approach depending on the viscosity of the sample. Suction may also be used, as will be further described below. Both approaches are expected to have acceptable volumetric accuracy. Once the sample portion is in the chamber, fluid communication between the chamber and the cup may be severed. The sample portion in the chamber may be isolated from the sample remaining in the cup by, for example, one-way (check) valves or by movement of the chamber away from the cup. A filter can be present between the cup and the chamber to filter out particles and sputum agglomerations from the sample portion.

In some embodiments additives may be added to aid in lysis of the bacteria cells, for pH control and the like. Possible additives include bacteria lysis reagents, buffers, detergents, tris-buffered saline, bovine serum albumen, pH modifiers and combinations thereof. Containment of the additive to mix with the sample portion requires intentional design considerations to address permeability of these contained additives for up to a 2 year shelf life. Minimizing leakage of such additives is important for volumetric accuracy (example: controlling a 4:1 additive/sample ratio) and also limiting water migration and evaporation. A film or foil laminate packet is one way to contain such additives. A packet, however, is difficult to adequately open so its contents can be fully emptied.

Venting issues associated with transfer of the packet's contents can also be problematic (e.g. using an elastomeric bladder). Additionally, the use of a check valve to seal an opening that ports to a confining structure around such additives not expected to provide an adequate permeability barrier. One approach is to use a fluid impermeable piston on each end of a relatively thick-walled cylindrical shell to store the additive. A rough estimate indicates that a 2% mm polypropylene or polyethylene confining wall around the additives could adequately maintain them over 3 years.

When an additive is provided to mix with and lyse the sample portion, up to a 5 minute delay may be anticipated prior to their delivery to the assay assembly. Additionally, if two additives or more are necessary, then the delay time could be different for each additive. Two suitable approaches for creating a delay are a mechanical timer or an electronic timer. A mechanical timer could be a clock type device (e.g., windup mechanism found in toys) or a restricted hydraulic/dampening feature (e.g., a viscous fluid flowing through an orifice). An example of an electronic timer is a microprocessor that is incorporated in the analysis module, and control of the timing is handled by software.

In some embodiments it may be desired to add more than one additive to the sample portion. In such case the additives may be incompatible or may degrade rapidly when mixed, so a single confining structure for both additives when mixed together may compromise shelf life preferences. For embodiments of this type a piston may be designed to deliver to the sample portion first one additive and then a second additive (or third, etc.). This sequential delivery of additives would avoid the problem of additive incompatibility.

An alternative to a confining structure for an additive is to apply the additive directly to a part of the assay assembly. For example, the additive could be applied as a liquid to, for example, a lateral flow assay strip, and allowed to dry. The dried residue of the additive would contact the sample portion when the sample portion was delivered and could then function in the same manner as additive that is mixed with the sample portion prior to contacting the assay assembly.

There are many reasonable approaches to transporting the sample portion and/or additives to the assay assembly. One embodiment involves movement of a piston to draw the sample portion into contact with the additive. If a film or foil seal is used to contain the additive, puncturing and venting must be integrated into the sequence. Additionally, establishing positive sealing during the transport of the sample portion is important. The actuation of the piston could be driven by a small motor (with a lead screw or rack and pinion), could be from releasing a spring, or could be by a manual push of the plunger handle. A permeable membrane may also be needed on the chamber to allow venting (but not allow liquid to escape).

One use of the device is to classify the sample as gram positive, gram negative or both when a suitable assay assembly is utilized. Exemplary assay assemblies include an enzyme-linked immunosorbent assay (ELISA) test, lateral flow assay, or flow through assay that may test for specific bacteria (Pseudomonas, Klebsiella, etc. . . . ), bacteria versus resistant strains (MRSA, Staph, etc. . . . ), specific proteins, viruses, molds, yeasts, fungi and enzymes.

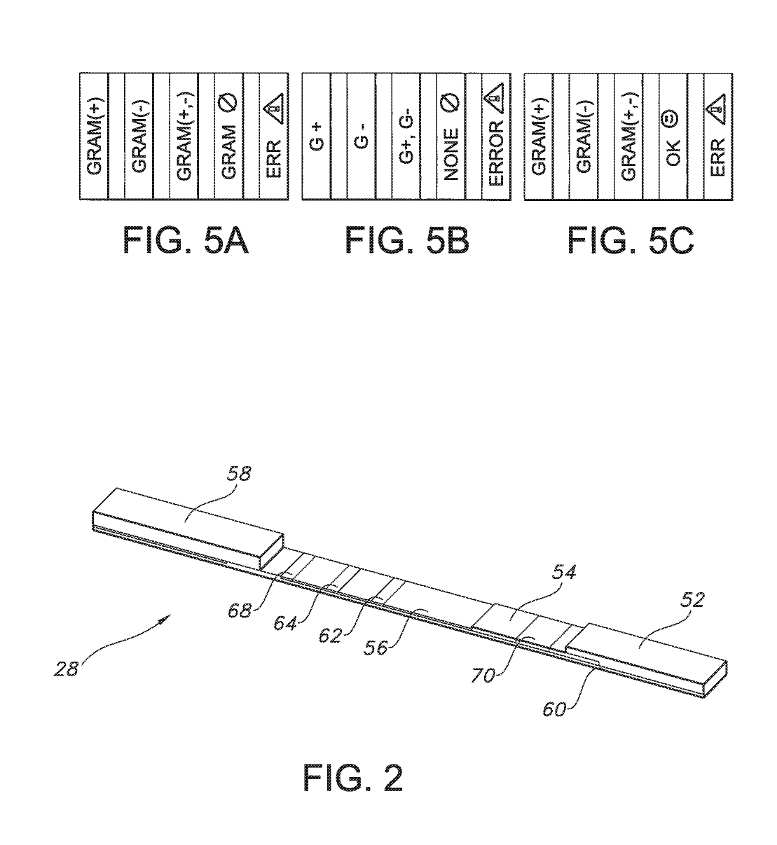

The ELISA test may desirably be a lateral flow assay test strip (LFA) that is an immunoassay (antibody detection) utilizing a visual (colorimetric) signal. The LFA employs a threshold detection system with a “positive” result when bacteria are above a 10³-10⁴ colony forming units/milliliter (cfu/ml). Colloidal gold, 40 nm, may be used as the detection label. Multiple analytes are used depending on the bacteria class; lipoteichoic acid (LTA) for Gram Positives and Lipopolysaccharide (LPS) for Gram Negatives. The LFA can utilize multi-line detection with between 2-4 test lines. The LFA usually includes one control line and may be direct antigen binding or a sandwich complex. A suitable additive is a running buffer consisting of tris-buffered saline with detergents (Tween 20) and non-specific proteins (bovine serum albumin or BSA) may be incorporated in the device. The detection and control lines are desirably read with reflectance-based measurements. The total time to run the test is desirably approximately fifteen minutes and desirably less than 30 minutes. The LFA desirably has the specifications given in Table 1. The item numbers in the left hand column of Table 1 may be found in FIG. 2, which shows a possible configuration for a lateral flow assay test strip.

TABLE 1

| Item | Description | Material |
|---|---|---|
| 52 | Sample Pad - may be | Cellulose |
| | eliminated in favor of | |
| | a larger conjugate pad. | |
| 54 | Conjugate Pad - may | Glass Fiber |
| | also receive the sample | |
| | if the Sample Pad is | |
| | eliminated. | |
| 56 | Detection Membrane | Nitrocellulose |
| 58 | Absorptive Sink | High Capacity Cellulose |
| 60 | Backing Card | Polycarbonate with adhesive on |
| | | one side |
| 62 | GP Test Line | Cocktail (or mixture) of anti-LTA |
| | | antibodies, monoclonal & |
| | | polyclonal, host animals: mouse, |
| | | goat, rabbit; Antibodies are |
| | | commercially available |
| 64 | GN Test Line | Cocktail of anti-LPS antibodies, |
| | | monoclonal & polyclonal, host |
| | | animals: mouse, goat, rabbit; |
| | | Antibodies are commercially |
| | | available |
| 66 | Control Line | Protein A |
| 68 | Conjugate, dried | Cocktail of anti-LTA and anti-LPS |
| | | antibodies conjugated to colloidal |
| | | gold particles. Additional |
| | | conjugated antibody for control line |
| | | is possible. |

The LFA will desirably be housed in a plastic enclosure that may be an integrated piece of the device. Fluid communication through the LFA is achieved through the physical overlap of discreet membranes. The physical contact between membranes may be maintained through plastic pinch points that may be part of the housing assembly. The sample may require additional processing steps prior to addition contact with the LFA. This processing could include the addition of bacteria lysis reagents, detergents, and other additives.

An alternate approach to processing the sample portion may include running the test with whole-cell, live bacteria. In this case an additive and mixing time are not needed and the sample only needs to be metered out of the cup in a known quantity prior to addition to the assay assembly.

Described below are a number of embodiments that demonstrate potential solutions. In addition to the system level concepts, accompanying solutions to more specific feature or components follow. It should be noted that one with ordinary skill in the art would understand that the system level and component level can, in many cases, be mixed matched, added, or eliminated depending on technical risk, feature requirements, and product cost targets. Also, it is possible that an additive will not be needed. In this case, the small test sample will still need to be acquired from the trap as before but could be presented to the assay assembly immediately, bypassing any step of mixing with an additive.

The following components may be found in the drawings.

Electronics—This section describes potential electronics and optics which can be utilized within in the device to sense a change in the test of the assay assembly and contribute towards the output or outputs that provide results of the test to the clinician. Electronics components found suitable for use in the device include a microprocessor, display 40, LEDs 22, detector 32, battery 36, and miscellaneous passive components (resistors and capacitors) and can all be located or attached to a common circuit board 34. These components are described individually below. These components were chosen because of their cost, size and basic suitability for the product
Microprocessor—The microprocessor runs the software that controls the LED 22, display 40, and detector 32. If an electronic means of controlling the timing of sample flow to the assay assembly is needed, the microprocessor will also control that function. The main characteristics are program memory, data memory, display control lines, LED control lines, and detector inputs. The microprocessor is located on the circuit board 34.
Display 40—The display's function is to provide information to the user on device status and assay assembly status, including indications of activity, error, gram positive, gram negative, and no bacteria present. The display can be a standard Twisted Nematic (TN) type liquid crystal display of size equivalent to those found in the pregnancy test products. The display could be driven directly by output pins on the microprocessor.
LED 22—The Light Emitting Diode provides illumination to the assay assembly. It must provide sufficient intensity for the photo-detectors to have sufficient signal-to-noise ratio to detect the indicators on the assay assembly. A suitable commercial LED is the SMT660 part.
Detector 32—The detector senses the change in an expression of a test line result in the assay assembly, such as reflected light, from the sensitive areas of the assay assembly. A suitable commercial photo-detector is the Advanced Photonix PDB-C154SM.
Battery 36—The battery must supply not only enough capacity to operate the “sleeping” device over its two plus years of shelf life, it must then supply sufficient current at a high enough voltage to drive the LED to its desired brightness. Small, long-life batteries (lithium coin cells) can provide significant life in a very small package but they tend to have a high internal resistance. This internal resistance manifests itself as a significant drop in voltage when large amounts of current are supplied to LED's or motors.
Motor—A motor may optionally be used to activate the wetting of the assay assembly. If so, this motor will be a small inexpensive DC motor such as commonly used in small toys.
Printed Circuit Board 34—The printed circuit board may be a two-sided board of approximately ˜2″×˜½″. The attachment of the LCD will vary with the chosen attachment method. This could be pinned, elastomeric (Zebrastrip), or heat seal.
Miscellaneous—A number of passive components (capacitors and resistors) would be needed in the design. These parts tend to be commodity items of very low cost.

The operation of the device can be divided into several phases. These are manufacturing, shelf life, monitoring, display results, and end-of-life. During the manufacturing phase, the device can provide information needed by the contract manufacturer to determine if the device was manufactured correctly. This may include turning on all the display icons, flashing the LED, etc. During shelf life, the device is in a very low power mode waiting for an input to indicate it is time to process the assay assembly. Once the input wakes up the device, it must activate the display and periodically turn on the LED and process results. This desirably occurs for about 15 minutes at which time the device transitions to the results phase. During the display results phase, the device shows the results on the display but no longer is running the detection circuitry. This is a low power mode that can last for considerable period of time. At some point, the device can transition to the end-of-life phase where it turns off the display.

Table 2 below shows an estimate of the energy needed to run the device across its operating life. It is assumed that the LED is operated at a 10% duty cycle (on for 100 ms per second) and is operated at 10 mA. It is also assumed the microprocessor is run at 1 MHz or less to reduce power. These assumptions result in a needed battery capacity of 30 mA-Hrs. This value can be reduced considerably by proper design of testing at manufacture (approximately 40% of needed capacity). In addition, the battery should be able to supply 10 mA for at least 100 ms without the voltage dropping below usable values (˜2.4V).

TABLE 2

| Stages of | | Micro | LCD | LED | Energy | |
|---|---|---|---|---|---|---|
| Operation | Time | mA | mA | mA | Used | |
| Manufacturing | 1 week | 0.07 | 0.001 | 0 | 11.928 | mA- |
| | | | | | | Hrs |
| Shelf Life | 2 years | 0.001 | 0 | 0 | 17.532 | mA- |
| | | | | | | Hrs |
| Monitoring | 15 minutes | 0.07 | 0.001 | 10 | 0.518 | mA- |
| | | | | | | Hrs |
| Display | 2 hours | 0.07 | 0.001 | 0 | 0.142 | mA- |
| Results | min. | | | | | Hrs |
| End Of Life | Per hour | 0.07 | 0.001 | 0 | 0.071 | mA- |
| | | | | | | Hrs |

| | | |
|---|---|---|
| Total capacity | 30.120 | mA- |
| | | Hrs |

The capacity of lithium coin cells is rated assuming a cutoff voltage of 2.0 V. Our device will need to operate above ˜2.4V (due to effect of pulse current) so the battery's capacity needs to be de-rated by 30% to account for this difference. The best choice for a lithium coin cell is the CR2025. It has a 160 mA-Hr capacity (de-rated to 112) and is capable of supplying pulse currents needed by the LED.

If a motor is needed for use in the device, it will require changes to the type of battery being used. Lithium coin cells have a high internal resistance which limits the current delivered by the battery and may prevent them from driving a motor. AAA-sized alkaline cells are a likely replacement for use with a motor. They can supply large amounts of current without significant drop in voltage. These cells will have considerably more energy than needed by the device. This may allow addition of a backlight if it is desired and the incremental cost increase is not excessive. Because of their naturally lower voltage than lithium (1.5 vs. 3.0), there will need to be two AAA batteries in the device.

### Example Embodiment 1

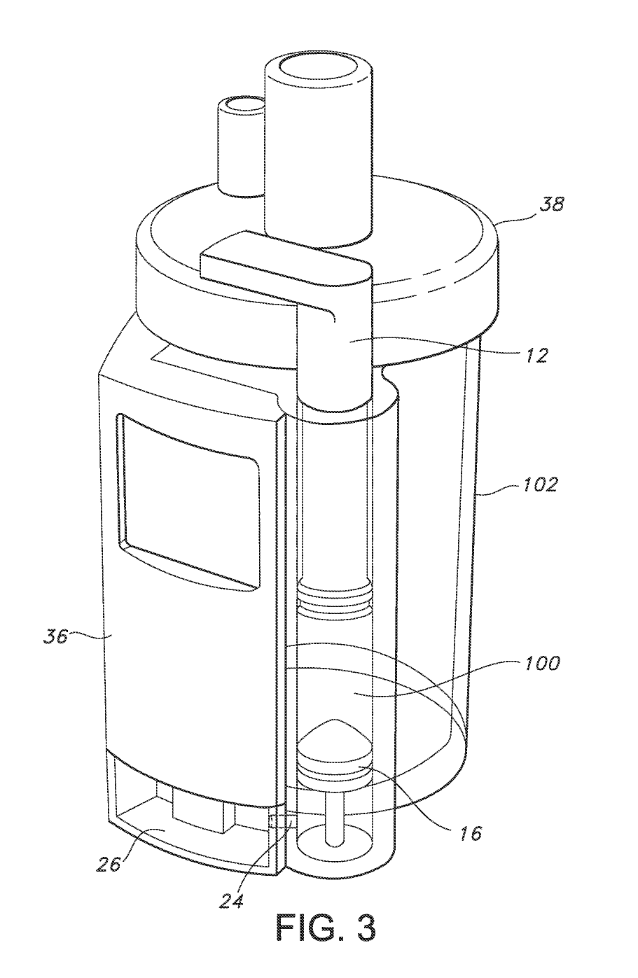

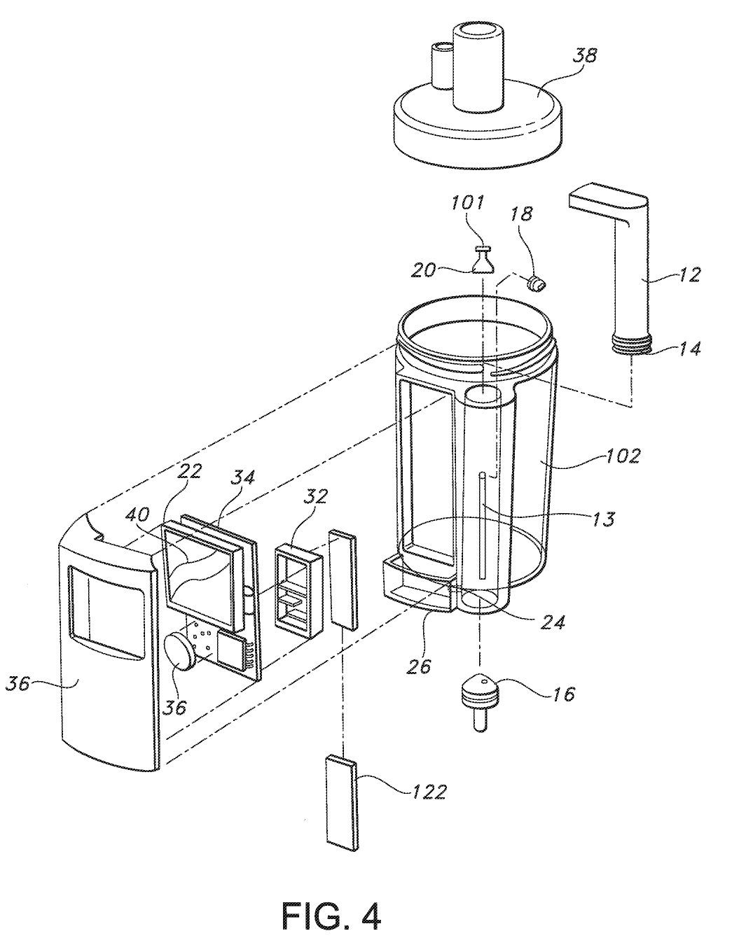

This embodiment utilizes one additive 20, one assay assembly 122, in this case a lateral flow assay (LFA) strip, and requires the user to manually apply effort to the device to move the sample, sample portion, and additive 20 to the appropriate positions within the device. An arrangement of two pistons, upper 14 and lower 16, conducts the sample portion and additive 20 towards the assay assembly 122. FIG. 3 shows a possible illustration of the embodiment. FIG. 4 demonstrates a possible exploded assembly view showing key components of the device.

Movement of one of the pistons draws the sample portion into contact with the additive 20. The movement of this piston could be the direct input of the user; e.g. lifting a plunger handle, or it could be driven by a small motor (with a lead screw or rack and pinion). The additive 20 is confined in a housing 101 of any shape designed for mixing with the sample portion. It should be noted that in this and the following embodiment, “pushing” the piston means that the piston is moved downwardly towards the cup 102 and “pulling” the piston means that the piston is moved upwardly away from the cup 102.

The medical user, i.e. a clinician, acquires a sample via current practice and the sample is deposited in the cup 102. The clinician may gently shake the device as needed to help make the sample homogeneous in the sample cup 102. The clinician will pull the handle 12 up, moving the upper piston 14 upwards and sucking the lower piston 16 with it as well as the additive 20 that is intitially confined in a space between the upper piston 14 and lower piston 16. Note that the space between the pistons defines the chamber 100. The movement of the lower piston 16 allows electrical contacts to touch, “waking up” the electronics. The lower piston 16 hits a hard stop at the same time the upper piston 14 moves past a check valve 18 inlet that it had been sealing closed. The suction force of the movement of the pistons pulls the sample portion up a conduit 13 from the bottom of the cup 102 through the check valve 18 and into the chamber 100 that confines the additive 20. If necessary, the clinician may gently shake the device to further mix the sample portion and additive 20. The clinician watches the display 22 until an indication is provided that the handle 12 can be depressed. When the handle 12 is pushed down by the clinician, the lower piston 16 is driven down until an outlet port 24 is uncovered, allowing the sample portion and additive 20 to flow into a well 26 where it comes in contact with the assay assembly 122. A port (not shown) is provided to allow any trapped air, but not liquid, to escape. A test result is displayed when the test is complete. The display can be as simple as LED lights or it can be the LCD screen display 22.

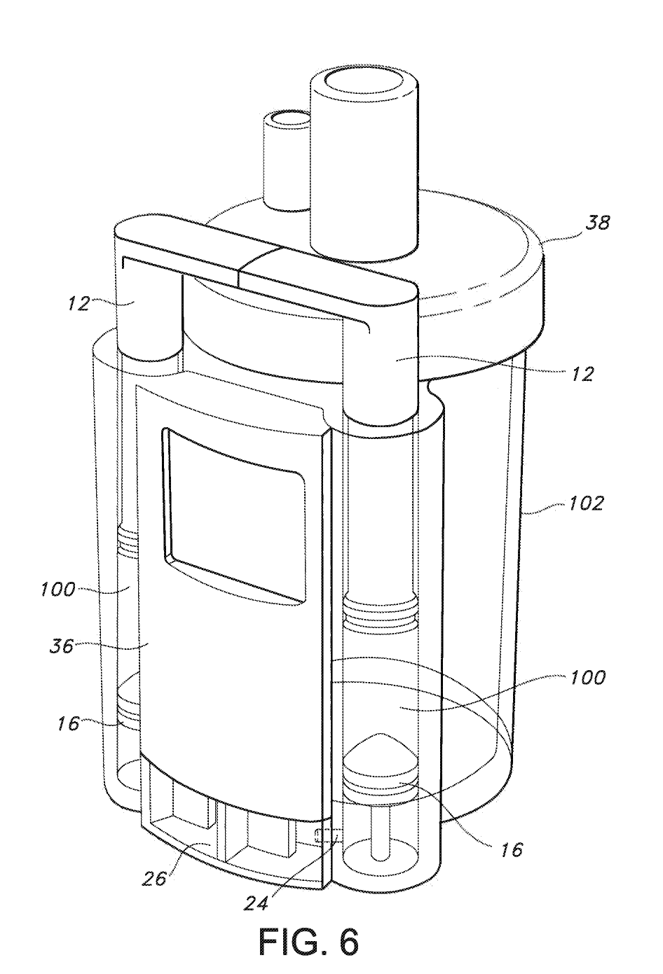

The display via the LCD screen may show the results of the test per the examples shown in FIGS. 5A, 5B and 5C but any option is appropriate. The examples of FIG. 5 indicate that the sample portion is gram negative, gram positive, both or neither over a set threshold amount, and includes a status if there is an error with the system. An error status can occur when the user fails to initiate a step within a given time period or if the sample fails to activate the ‘control line’ on the assay assembly. In FIGS. 5A, 5B and 5C different ways of expressing the results are shown. Each figure indicates a gram positive result on the far left, followed by gram negative, both gram positive and negative present, neither gram positive nor negative present, and, on the right, an error signal.

### Example Embodiment 2

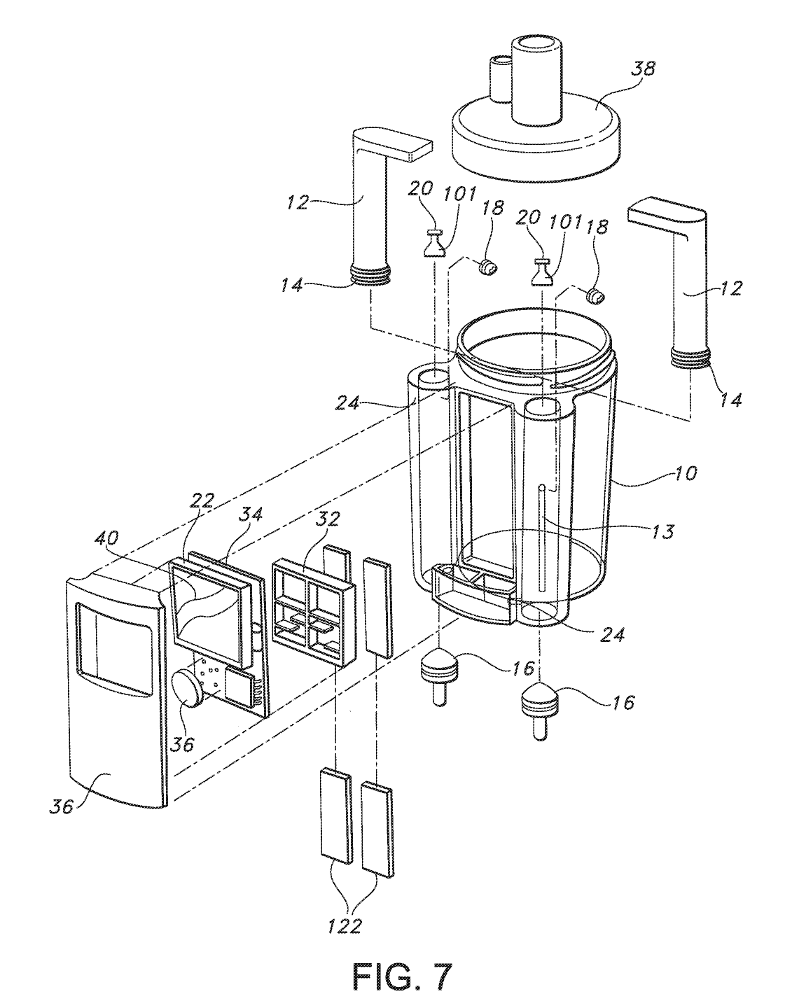

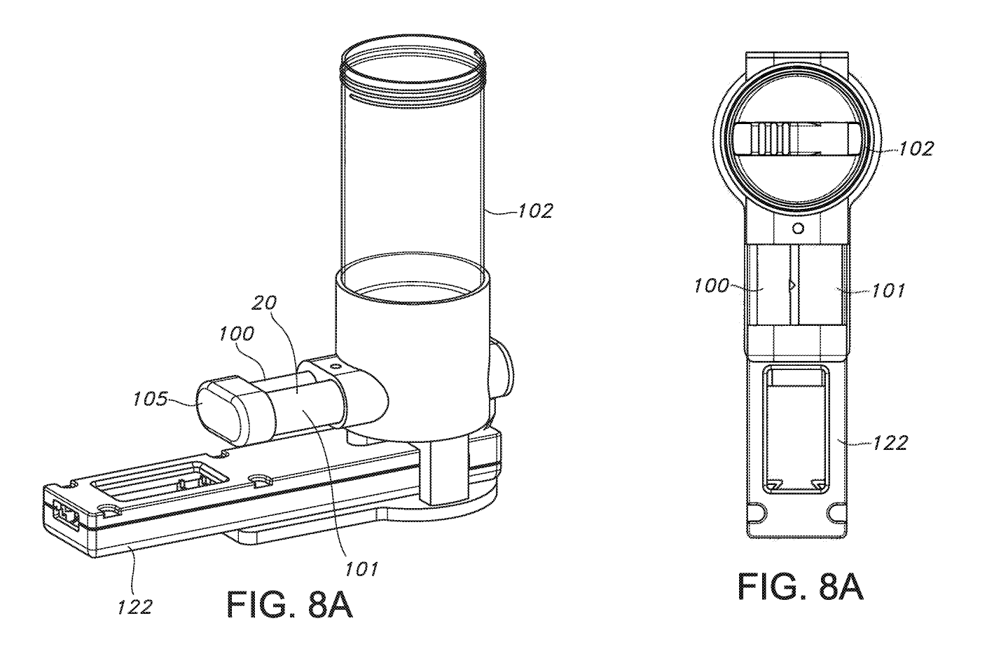

The second embodiment to be described is more complicated and requires the incorporation of two tests within the assay assembly or two assay assemblies 122, one for Gram Positive (GP) and one for Gram Negative (GN), and two liquid additives 20, one for each assay. FIG. 6 is a drawing of an embodiment of such features integrated into an exemplary device. FIG. 7 is an exploded view of the embodiment of FIG. 6.

In this system, the user acquires a sample and manipulates the device, similar to Embodiment 1. The clinician will pull the handles 12 up, moving the upper pistons 14 upwards, sucking the additives 20 and lower pistons 16 with them. Note that the handles 12 may be moved simultaneously or sequentially. Subsequent actions within the device proceed in the same manner as described for Embodiment 1: one set of actions proceeds for movement on one of the handles 12; another set of actions proceed for movement of the other handle 12.

An alternative to the vertical orientation of embodiments 1 and 2 is a horizontal orientation. In a horizontal orientation, the check valve can be located in the bottom of the cup, for example in a well, to minimize entrapped air and sample volume and avoid starvation/clogging of the conduit 13 that the sample portion follows to the assay assembly 122. Air trapped must be managed to ensure the correct amount of fluid is delivered to the assay assembly 122 while remaining liquid tight. There will be air trapped above the two ports and some additional air may come in to replace liquid vapor losses.

### Example Embodiment 3

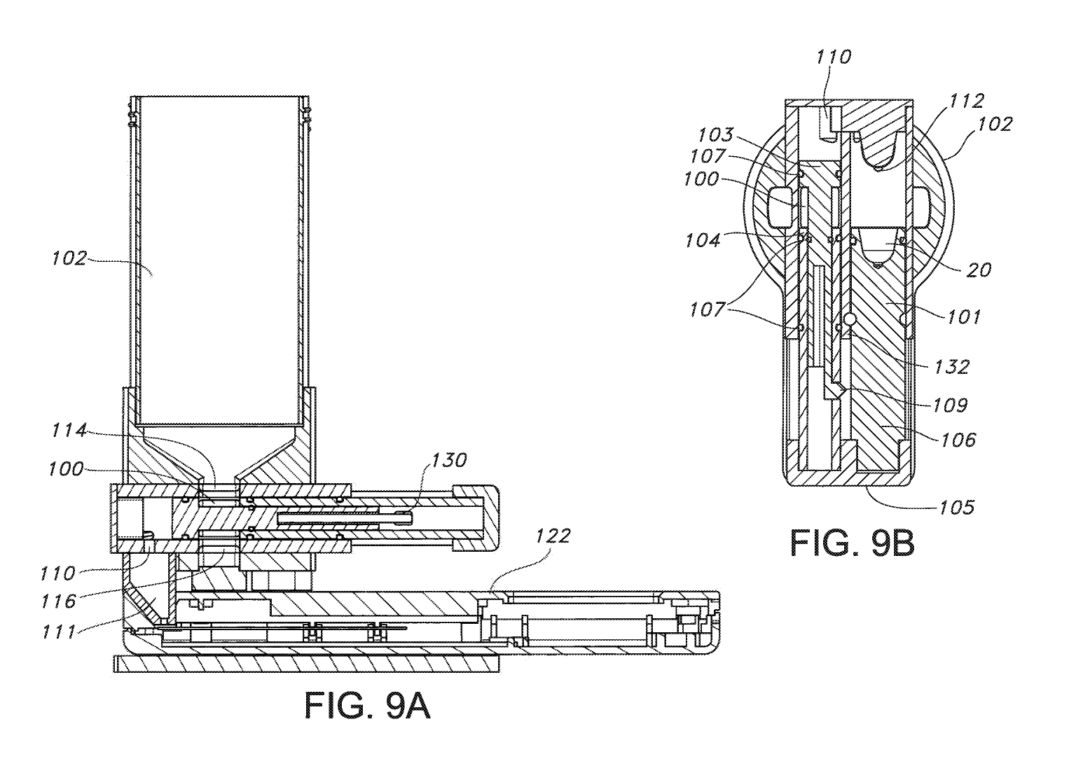

Yet another embodiment is shown in FIGS. 8A and 8B. FIG. 8A is a top view of the device and FIG. 8B is a side perspective view. This device includes two horizontal containment structures, generally in the form of side-by-side cylinders; one for providing the sample chamber 100 and the second a housing 101 to initially confine an additive 20 and/or additives. There is also a collection cup 102 which is initially in fluid communication with the sample chamber 100 so that a portion of the sample that has been collected in the cup 102 may be positioned within the chamber 100. As shown in FIG. 8, the cup 102 may be generally cylindrical. The sample chambers 100 and housing 101 are shown generally perpendicular to the cup 102, though this geometrical arrangement is not required.

In the start position, a portion of the sample chamber 100 is open to the cup 102 via top and bottom openings 114, 116 in the sample chamber 100 (FIGS. 9A and 9B).

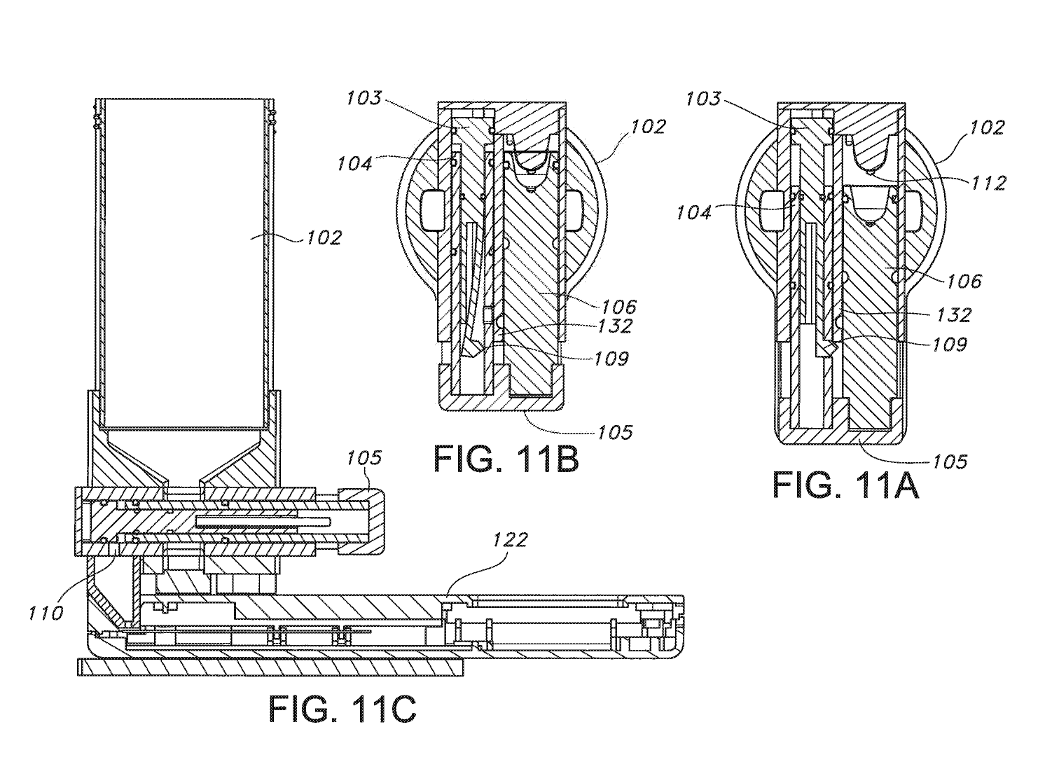

Sample collection may be done in one step or may be done serially. The sample(s) that is introduced into the cup 102 is allowed to flow and mix through/into the chamber 100. By placing the chamber 100 towards the bottom of the cup 102, a minimum portion of sample (e.g. less than 5 cc) is required for the device to function correctly. This design also allows for use with a wide variation of sample characteristics, from low to high viscosities to samples with and without included particulates. A filtration media (e.g. ribs) 118 may be placed around the sample chamber 100 to enable a gross level of filtering of large particles and high viscosities. FIG. 10 depicts a suitable arrangement of ribs that serve as the filtration media around the chamber 100. A foam pad material may also be placed within a well 111 to further filter the sample portion and any additive 20 and/or additives.

The chamber 100 is open to the cup 102, where the opening may be via two entrances, multiple entrances, or even an unbounded entrance. Desirably, the chamber 100 is open to the cup 102 at the top and bottom so that sample may flow into the chamber 100 freely. Having multiple open pathways for the sample to enter the chamber 100 ensures filling of the chamber and helps prevent formation of an air pocket or pockets within the chamber. The chamber 100 is positioned between movable walls respectively joined to movable pistons 103, 104. These pistons are in communication with each other and also with an activation button (push button) 105. O-rings 107 or other means may be used to maintain a liquid seal between the pistons 103, 104 and the surrounding walls. It should be noted that in this and the following embodiments, “pushing” the button 105 means that the button 105 is moved towards the cup 102 and “pulling” the button 105 means that the button 105 is moved away from the cup 102.

Prior to use, the two pistons 103, 104 are locked a set distance from each other, creating a spacing (the chamber 100) that dictates the volume of the sample portion to be isolated (FIG. 9B). The housing 101 may serve as a movable piston or there may be a separate piston 106 which is also in communication with the push button 105. The additive 20 and/or additives are located in this housing 101 between the piston 106 and the outlet port 110 to the assay assembly or assemblies 122.

In a general configuration, the additive 20 and/or additives can be contained in blister packs or the like. For example, the additive 20 and/or additives may be located inside the piston 106 and sealed in a puncture-able package. The package may be made from or sealed with a film or foil which may desirably be metallic to reduce permeability issues (e.g. leakage).

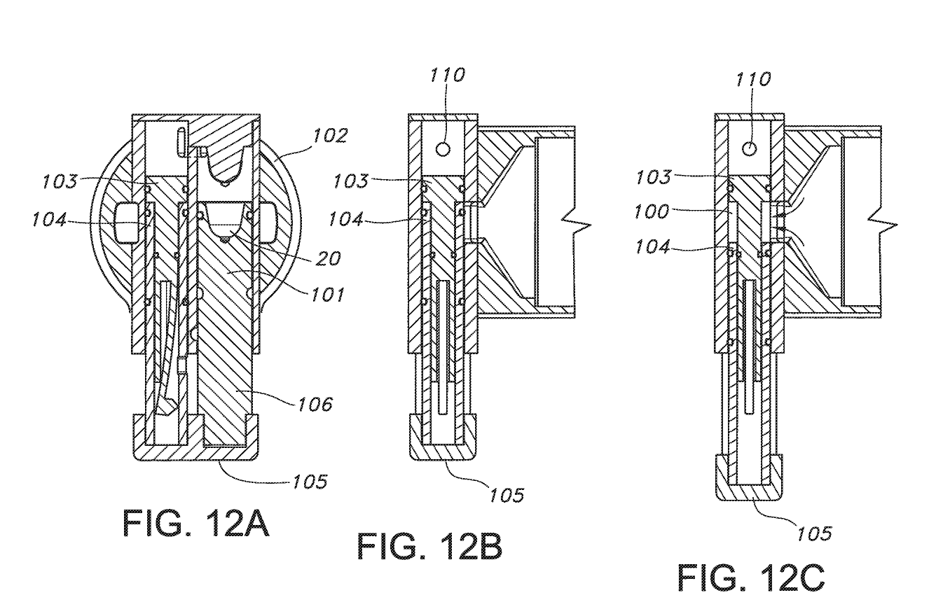

After a sample portion has been collected in the chamber 100, the device may be activated by the user pushing the button 105 in completely. With this one motion the following steps occur within the chamber 100 and housing 101:
1) the pistons 103, 104 slide forward, conducting the sample portion from the chamber 100 toward the well 111 and isolating the sample portion from the rest of the sample remaining in the cup 102 (see FIG. 11A); simultaneously, the additive piston 106 moves forward and moves the additive toward the end of its stroke where it will be released. If the additive is in a package the package will be punctured at the end of the stroke by a point 112. Once the sample portion is completely isolated from the chamber 102, an inner locking mechanism tab 109 which keeps the two pistons spaced a set distance apart is pushed in by the end of a rod 132 and the two pistons 103, 104 become unlocked from each other.
2) As the leading piston 103 reaches the end of its stroke the trailing piston 104 moves forward and pushes the isolated sample portion through the exit port 110 to the strip well 111 (FIGS. 11B and 11C). Simultaneously, the additive piston 106 hits the end of its stroke where the package is punctured by contact with a point 112 and the additive 20 and/or additive content is pushed towards the well 111 through the exit port(s) 110. Both the sample portion and the additive 20 exit their respective chambers 100, 101 via connected, common port or ports 110 and come together, desirably mixing effectively, prior to reaching assay assembly 122 and desirably prior to the well 111

It is desirable that chamber 100 and housing 101 are relatively completely evacuated by the movement of the appropriate pistons, allowing all the fluid to be injected to the well 111 (FIG. 11A). It should be noted that FIG. 9B shows the device in cross section prior to activation and FIG. 11B shows the device in cross section almost completely activated. (FIG. 11B shows the device prior to complete activation for clarity.)

Effective mixing is enhanced by desirably having both the sample portion and additive 20 and/or additives start and stop flowing though the common port 110 at the same time. Implicit in proper design of the device is pre-determining appropriate volume and ratio of sample portion to additive 20 and/or additives for use by the assay assembly or assemblies. Piston stroke lengths and internal dimensions of the sample chamber, the containment structure, associated conduits, ports, well, etc. must also be properly designed.

In other embodiments, the sample portion and additive 20 and/or additives can be delivered to more than one assay assembly 122 depending on test requirements. The inclusion of the well 111 can interface with more than one assay assembly. Each assay assembly 122 can each have a port coming from the well 111. It is believed that the device can apply a sufficient amount of sample portion with additive 20 and/or additives to the assay assembly or assemblies 122 even when the device is activated in a non-neutral position; e.g. a 40 degree tilt. This is accomplished by sizing the sample portion, chamber 100, housing 101, etc. appropriately to obtain sufficient volume of the sample portion with additive 20 and/or additives in the well 111.

### Example Embodiment 4

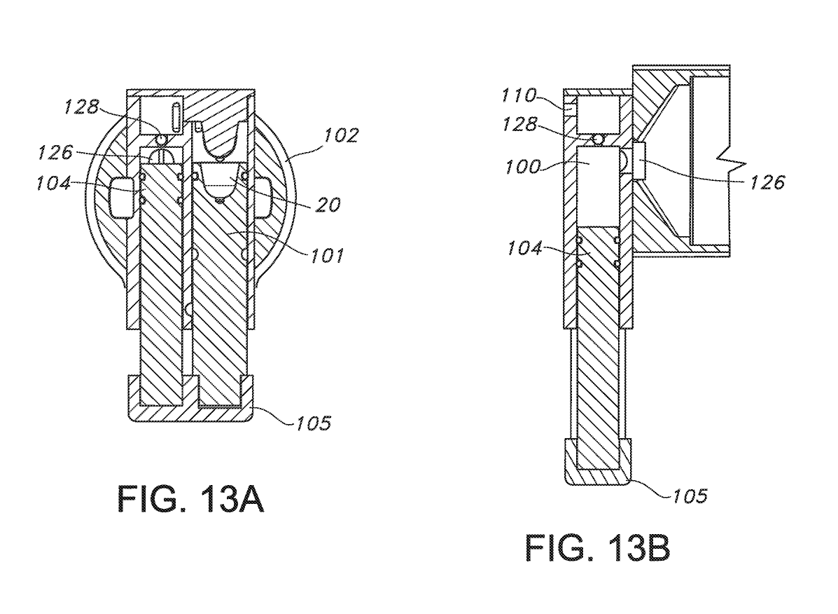

This embodiment is similar to Embodiment 3 except this embodiment has the ability to actively fill the sample chamber 100 with a sample portion from the cup 102. This is accomplished with the trialing piston 104 being initially placed adjacent to the leading piston 103 prior to user activation as shown in FIGS. 12 A and 12B. The additive 20 is present in a housing 101. FIG. 12A shows a top view of the device with the trailing piston 104 adjacent the leading piston 103. The additive 20 is also shown. The same position is shown in a cutaway side view in FIG. 12B with the pistons 103, 103 adjacent to each other and the exit port 110 visible. The first step of user activation is to pull out on the button 105 as shown in FIG. 12C. As the trailing piston 104 moves away from the leading piston 103 a vacuum is generated. The vacuum pulls a sample portion from the cup 102 into the space between the pistons 103, 104, i.e., the sample chamber 100, with which it is in fluid communication. From this point forward the embodiment is identical to Embodiment 3.

### Example Embodiment 5

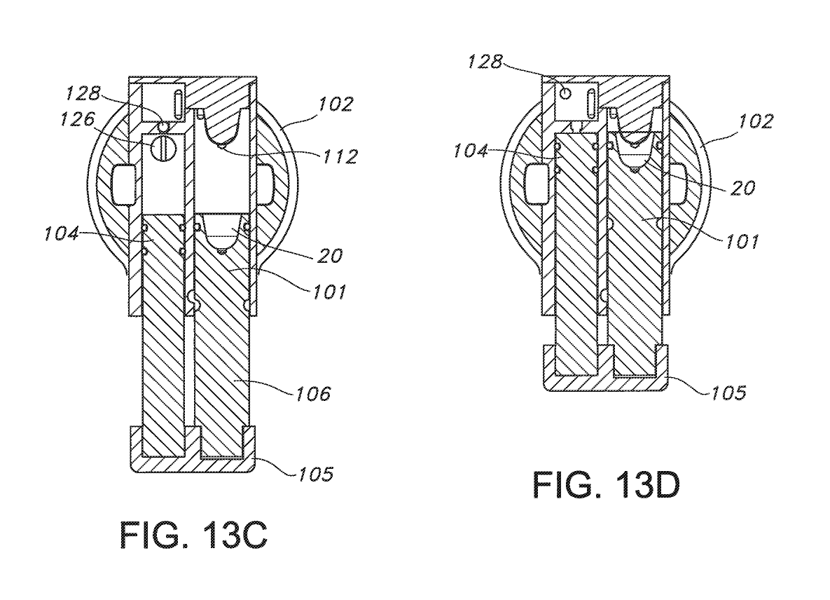

This embodiment is similar to Embodiment 4 except this embodiment replaces leading piston 103 with a set of one way check valves. FIG. 13A shows a bottom view of the device showing the trailing piston 104 and the first check valve 126. Prior to activation, the sample chamber 100 does not contain any sample and is separated from the cup 102 by the first check valve 126 that is in fluid communication with the cup 102 and only allows a sample portion to flow into the chamber 100 when the piston 104 is pulled back (FIG. 13B). When the piston 104 is pulled back a sample portion is drawn into the chamber 100 from the cup 102 through the first check valve 126 as shown in FIG. 13C.

A second check valve 128 only allows an isolated sample portion to flow out of the chamber 100 to the exit port 110 as illustrated in FIG. 13D.

As in embodiment 4, as the user pulls out on the button 105, the trailing piston 104 moves outward and the sample portion from the cup 102 flows through the first check valve 126 and fills the sample chamber 100, with which it is in fluid communication, because of the vacuum generated between the pistons. After the chamber 100 is filled, the user pushes the button 105, the trailing piston 104 moves forward and the sample portion exits the chamber 100 through the second check valve 128 to the exit port 110 where it mixes with the additive 20 as in Embodiments 3 and 4. The first check valve can be a duckbill valve, umbrella valve, spring loaded valve or any another type of one-way valve that operates similarly. The second check valve 128 may any of those suitable for the first check valve 126 and may also be a simple plug or ball that is able to pop open when a pressure is generated when the trailing piston 104 advances forward. The additive 20 is present in a housing 101.

### Example Embodiment 6

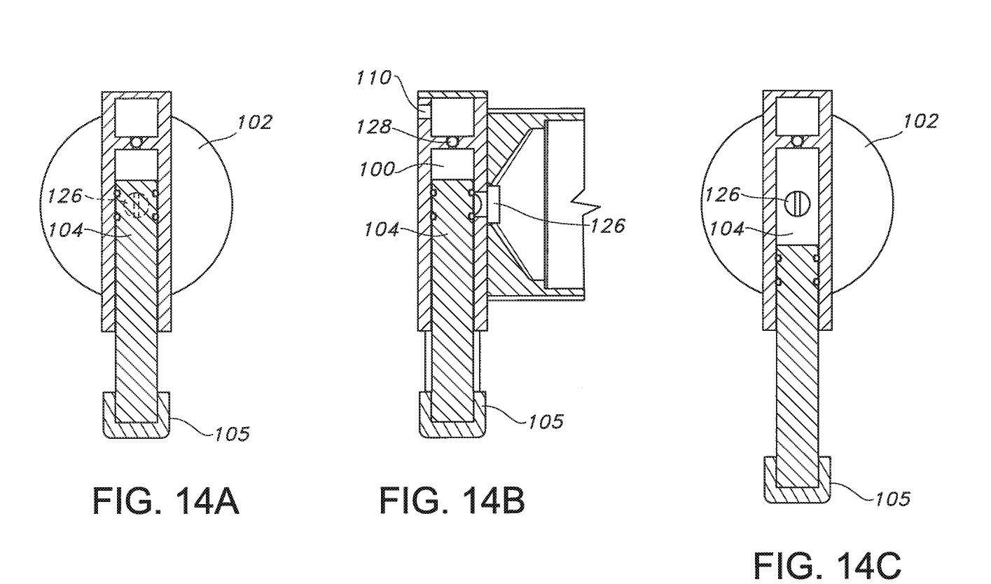

This embodiment is similar to Embodiment 5 except that this embodiment does not have a housing 101 for the additive 20. In this embodiment, the additive 20 is initially housed in the sample chamber 100. FIG. 14A shows a cutaway top view of the device and FIG. 14B shows a cutaway side view in the initial position prior to activation. As the user activates the device by pulling the button 105, the trailing piston 104 moves outwardly, generating a suction that pulls a sample portion into the chamber 100 through the first check valve 126 from the cup 102 (FIG. 14A). The sample portion mixes with the additive 20 already in the chamber 100 as it enters. As in Embodiment 5, as the user pushes in the button 105, the sample portion mixed with the additive 20 are pushed out through the second check valve 128 towards the exit port 110.

As used herein and in the claims, the term “comprising” is inclusive or open-ended and does not exclude additional unrecited elements, compositional components, or method steps.

While various patents have been incorporated herein by reference, to the extent there is any inconsistency between incorporated material and that of the written specification, the written specification shall control. In addition, while the disclosure has been described in detail with respect to specific embodiments thereof, it will be apparent to those skilled in the art that various alterations, modifications and other changes may be made to the disclosure without departing from the spirit and scope of the present disclosure. It is therefore intended that the claims cover all such modifications, alterations and other changes encompassed by the appended claims.

What is claimed is:

1. A device to test a sample, the device comprising; a cup for receiving a sample, a chamber in fluid communication with said cup for receiving and defining a portion of the sample from the cup, and a piston for moving said sample portion from said chamber to an assay assembly.

2. The device of claim 1 wherein said sample is sputum collected from a patient.

3. The device of claim 2 wherein said sample is collected from a lung of said patient.

4. The device of claim 1 wherein a motion of said piston isolates said sample portion from said cup.

5. The device of claim 1 further comprising a first additive that is added to said sample portion prior to delivery to said assay assembly.

6. The device of claim 1 further comprising a first additive present as a dried residue in said assay assembly.

7. The device of claim 5 wherein said additive is added to said sample portion by a motion of a second piston, locked to the first piston.

8. The device of claim 5 wherein a second additive is added to said sample portion after said first additive.

9. The device of claim 7 wherein said first and second pistons become unlocked from each other once the sample portion is completely isolated from the chamber.

10. The device of claim 5 wherein said sample portion and additive mix in a common port prior to contacting said assay assembly.

11. The device of claim 5 wherein said first additive is selected from the group consisting of bacteria lysis reagents, detergents, tris-buffered saline, bovine serum albumen, pH modifiers and combinations thereof.

12. The device of claim 1 further comprising an assay assembly that performs at least one test on the sample portion.

13. The device of claim 12 wherein said test comprises detection of gram positive bacteria, or gram negative bacteria, or both gram positive and negative bacteria.

14. The device of claim 1 further comprising a filter between said cup and said chamber.

15. The device of claim 1 further comprising a detector that senses a change in reflected light from the assay assembly.

16. The device of claim 15 further comprising a display that receives information regarding the change in reflected light from the detector and displays said information to a user.

17. The device of claim 1 wherein said assay assembly is an enzyme-linked immunosorbent assay.

18. The device of claim 17 wherein said immunosorbent assay is a lateral flow assay.

19. The device of claim 1 where the chamber has more than one discrete opening to the cup.

20. The device of claim 1 where the chamber has an unbounded opening to the cup.
